(19) United States
(12) Patent Application Publication
(10) Pub. No.: US 2023/0277268 A1
(43) Pub. Date: Sep. 7, 2023

(54) ARM DRAPE ASSEMBLY FOR ROBOTIC SURGICAL SYSTEM

(71) Applicant: Cilag GmbH International, Zug (CH)

(72) Inventors: Kandice Hoi-Ning Lau, San Francisco, CA (US); Richard M. Lum, San Jose, CA (US)

(21) Appl. No.: 18/116,880

(22) Filed: Mar. 3, 2023

Related U.S. Application Data
(60) Provisional application No. 63/317,286, filed on Mar. 7, 2022.

(51) Int. Cl. A61B 46/10; A61B 34/30

(52) U.S. Cl. CPC A61B 46/10; A61B 34/30; A61B 2034/305

(57) ABSTRACT

An assembly includes a first sterile adapter and a sterile drape. The first sterile adapter is configured to be received by a first movable robotic arm of a robotic surgical system. The sterile drape includes a first sleeve configured to extend over the first movable robotic arm. The first sleeve includes a first end, a second end disposed opposite the first end, and an intermediate portion disposed between the first and second ends. The first end is coupled with the first sterile adapter. The first end is inverted toward the second end. The intermediate portion includes a first roll.

### PRIORITY

This application claims priority to U.S. Prov. Pat. App. No. 63/317,286, entitled “Arm Drape Assembly for Robotic Surgical System,” filed Mar. 7, 2022, the disclosure of which is incorporated by reference herein, in its entirety.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, holding or driving a needle, grasping a blood vessel, dissecting tissue, or cauterizing tissue.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of a table-based robotic system configured for a laparoscopic procedure;

FIG. 2 depicts a perspective view of a second example of a table-based robotic system;

FIG. 3 depicts an end elevational view of the table-based robotic system of FIG. 2;

FIG. 4 depicts the end elevational view of the table-based robotic system of FIG. 3 including an example of a pair of robotic arms;

FIG. 5 depicts a partially exploded perspective view of the robotic arm of FIG. 4 having an instrument driver and a first example of a surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7 depicts an example of a plurality of robotic arms configured to move along an adjustable arm support for use with the table-based robotic system of FIG. 4;

FIG. 8 depicts an example of a sterile drape assembly in a deployed configuration over the robotic arms and adjustable arm support of FIG. 7, with portions of the frame assembly hidden;

FIG. 9 depicts a perspective view of the sterile drape assembly of FIG. 8 in a packaged configuration, with the sterile drape hidden;

FIG. 10 depicts a top view of the sterile drape assembly of FIG. 9;

FIG. 11 depicts a side elevational view of the sterile drape assembly of FIG. 9;

FIG. 12A depicts a schematic cross-sectional side elevational view of a sterile drape of the sterile drape assembly of FIG. 7 after a first manufacturing stage;

FIG. 12B depicts a schematic cross-sectional side elevational view of a sterile drape of the sterile drape assembly of FIG. 7 after a second manufacturing stage;

FIG. 12C depicts a schematic cross-sectional side elevational view of a sterile drape of the sterile drape assembly of FIG. 7 after a third manufacturing stage;

FIG. 12D depicts a schematic cross-sectional side elevational view of a sterile drape of the sterile drape assembly of FIG. 7 after a fourth manufacturing stage;

FIG. 12E depicts a schematic cross-sectional side elevational view of a sterile drape of the sterile drape assembly of FIG. 7 after a fifth manufacturing stage;

FIG. 12F depicts a schematic cross-sectional side elevational view of a sterile drape of the sterile drape assembly of FIG. 7 after a robotic arm partially unrolls the sterile drape;

FIG. 13 depicts an enlarged schematic sectional view of a portion of the sterile drape assembly of FIG. 9, with an example of a locking assembly in the closed configuration, with the open configuration being shown in phantom;

FIG. 14 depicts a bottom view of the locking assembly of FIG. 13;

FIG. 15 depicts a plan view of another example of a locking assembly that may be used with the sterile drape assembly of FIG. 9;

FIG. 16A depicts a perspective view of the sterile drape assembly of FIG. 8 placed over the robotic arms of FIG. 7;

FIG. 16B depicts a perspective view of the robotic arms and the sterile drape assembly of FIG. 16A, but with the sterile drape assembly being moved over the robotic arms toward the adjustable arm support of FIG. 7;

FIG. 16C depicts a perspective view of the robotic arms, the adjustable arm support, and the sterile drape assembly of FIG. 16B, but with a portion of the sterile drape assembly being moved toward a first end of the adjustable arm support;

FIG. 16D depicts a perspective view of the adjustable arm support and sterile drape assembly of FIG. 16C, but with a portion of the sterile drape assembly being rotated over the first end of the adjustable arm support; and

FIG. 16E depicts a perspective view of the sterile adapter of the sterile drape assembly of FIG. 16A being coupled with the instrument driver of the robotic arm of FIG. 16A.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

FIG. 1 shows an example of a robotically-enabled medical system, including a first example of a table-based robotic system (10). Table-based robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of table-based robotic system (10) may be constructed and operable in accordance with at least some of the teachings of any of the various patents, patent application publications, and patent applications that are cited herein.

A. Example of Table-Based Robotic System with Annular Carriage

With respect to FIG. 1, table-based robotic system (10) includes table system (12) having a platform, such as a table (16), with a plurality of carriages (18) which may also be referred to herein as “arm supports,” respectively supporting the deployment of a plurality of robotic arms (20). Table-based robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

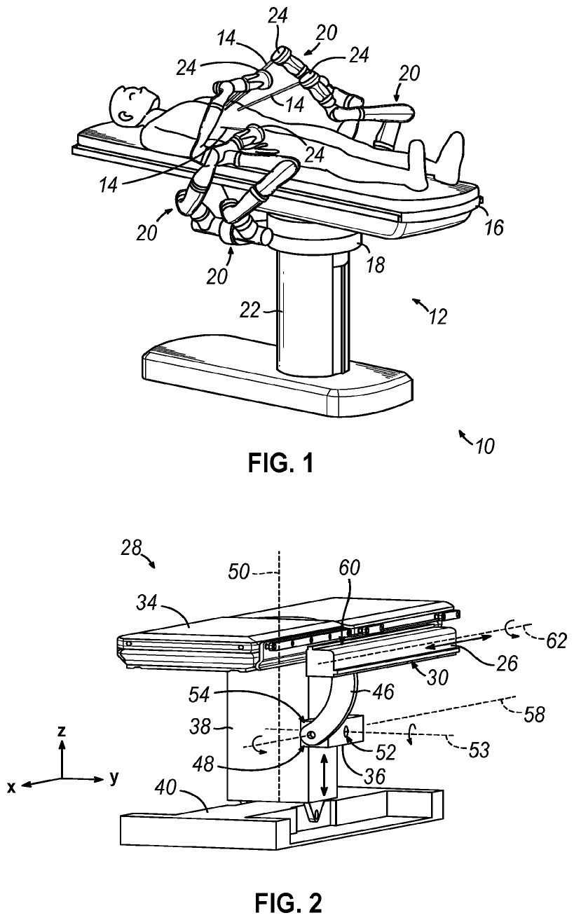

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, table-based robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Table-based robotic system (10) may also include a tower (not shown) that divides the functionality of table-based robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

B. Example of Table-Based Robotic System with Bar Carriage

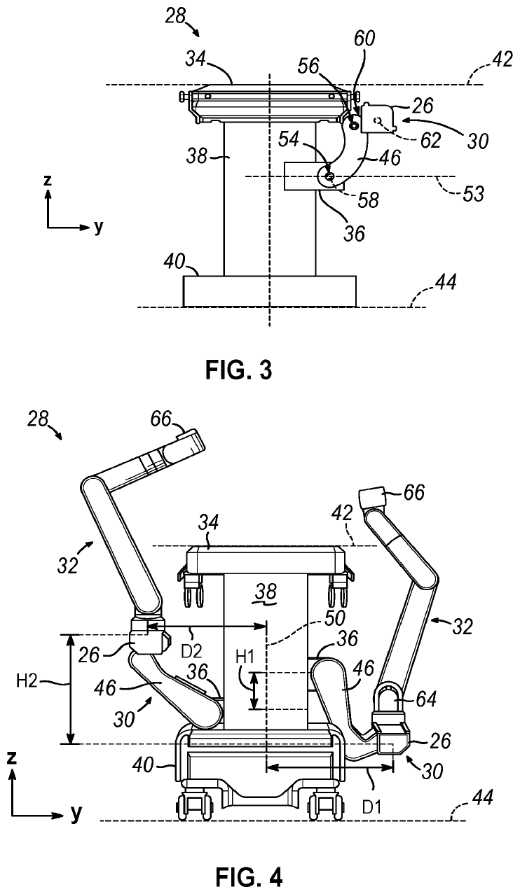

FIGS. 2-4 show another example of a table-based robotic system (28). Table-based robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to table-based robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of table-based robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

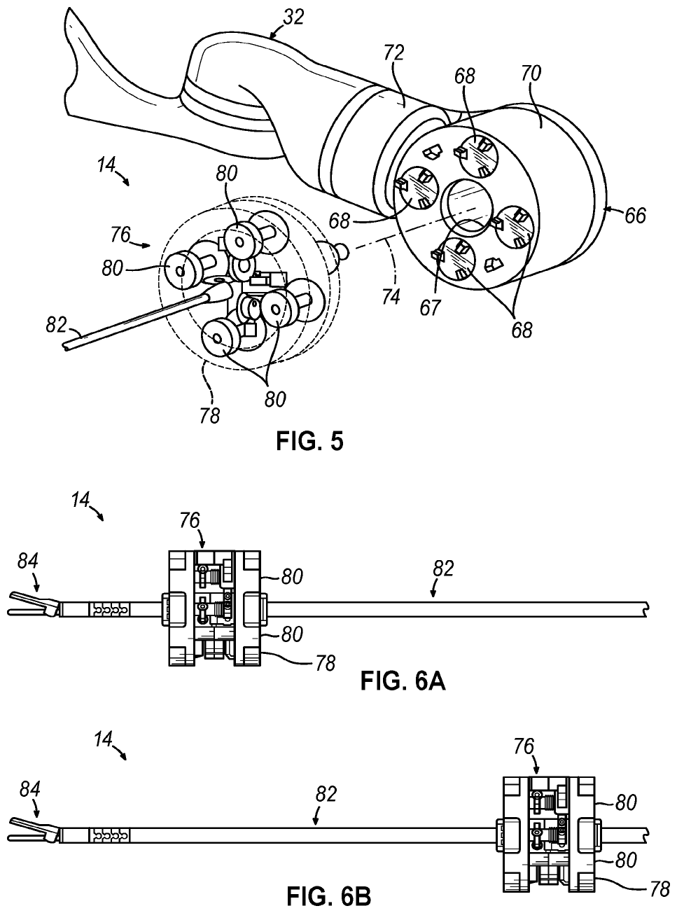

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

C. Example of Surgical Instrument with Instrument-Based Insertion Architecture

FIGS. 5-6B show surgical instrument (14) having the instrument-based insertion architecture as discussed above. Surgical instrument (14) includes elongated shaft assembly (82), end effector (84) connected to and extending distally from shaft assembly (82), and instrument base (76) coupled to shaft assembly (82). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

As shown in FIGS. 5-6B, and in cooperation with instrument driver (66) discussed above, surgical instrument (14) includes an elongated shaft assembly (82) and an instrument base (76) with an attachment interface (78) having a plurality of drive inputs (80) configured to respectively couple with corresponding drive outputs (68). Shaft assembly (82) of instrument (14) extends from a center of instrument base (76) with an axis substantially parallel to the axes of the drive inputs (80) as discussed briefly above. With shaft assembly (82) positioned at the center of instrument base (76), shaft assembly (82) is coaxial with instrument driver axis (74) when attached and movably received in clearance bore (67). Thus, rotation of rotational assembly (70) causes shaft assembly (82) of surgical instrument (14) to rotate about its own longitudinal axis while clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

### II. Example of Robotic Arm Assembly and Corresponding Sterile Drape Assembly

With robotic arms (20, 32) being positioned near a patient during a robotic surgical procedure, at least a portion of each robotic arm (20, 32) may enter the sterile field defined for the surgical procedure. It may therefore be desirable to provide robotic arms (20, 32) in a sterile state to prevent robotic arms (20, 32) from compromising sterility in the sterile field. In addition, in some robotic procedures, bodily fluids and other debris may tend to reach robotic arms (20, 32), such that it may be desirable to shield robotic arms (20, 32) from such fluids and debris. The size and configuration of robotic arms (20, 32) may also make it difficult to clean and appropriately sterilize robotic arms (20, 32) between surgical procedures. It may therefore be desirable to provide a removable drape assembly that may be used to cover robotic arms (20, 32) during robotic surgical procedures. Such drape assemblies may be sterile, thereby providing a barrier that prevents robotic arms (20, 32) from otherwise contaminating sterility of a sterile field during a robotic surgical procedure. Such drape assemblies may also protect robotic arms (20, 32) from bodily fluids and other debris that might otherwise reach robotic arms (20, 32) during a robotic surgical procedure. Such drape assemblies may thus be removed at the end of the robotic surgical procedure, reducing the time and effort needed to clean robotic arms (20, 32) between robotic surgical procedures.

Examples of sterile drapes are shown and described in U.S. Pub. No. 2021/0153965, entitled “Systems and Methods for Draping a Surgical System,” published on May 27, 2021; U.S. Pub. No. 2021/0153966, entitled “Drape for Arms of a Robotic Surgical System,” published on May 27, 2021; the disclosure of each which is incorporated by reference herein, in its entirety. An additional example of a drape assembly is described in greater detail below. An example of a robotic arm assembly, with which the drape assembly may be used, is also described in greater detail below.

A. Example of Robotic Arm Assembly

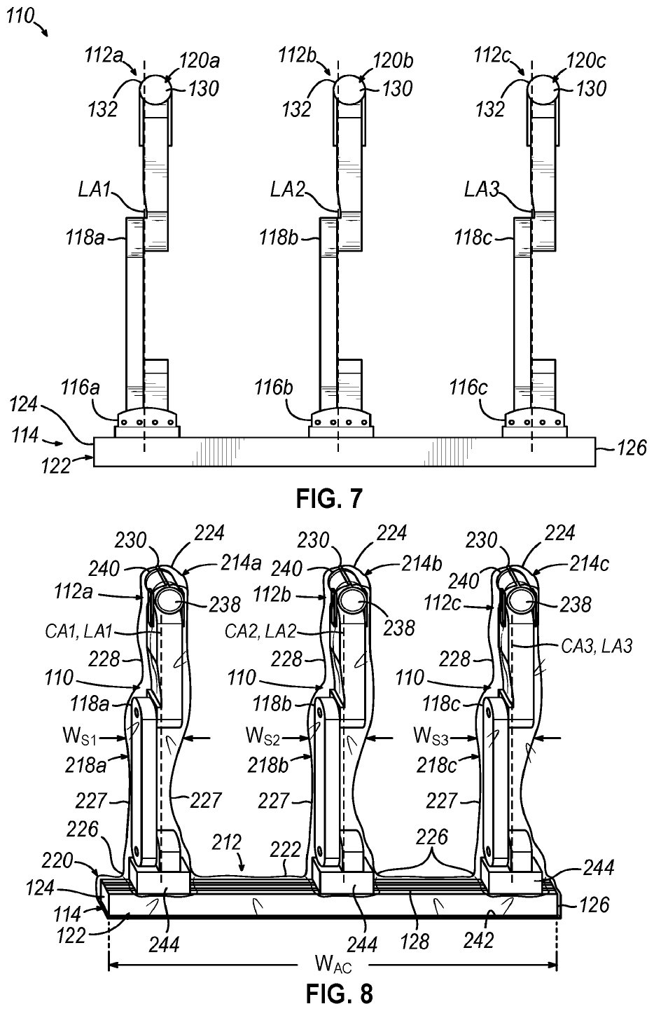

FIG. 7 shows an example of a robotic arm assembly (110) that includes a plurality of robotic arms (shown as first, second, and third robotic arms (112a-c)) and an adjustable arm support (114). First, second, and third robotic arms (112a-c) may be used in place of robotic arms (32); and adjustable arm support (114) may be used in place of adjustable arm support (30) of FIG. 4. First, second, and third robotic arms (112a-c) may be operatively coupled with adjustable arm support (114). While first, second, and third robotic arms (112a-c) are shown, more or fewer robotic arms are envisioned. First, second, and third robotic arms (112a-c) may be the same or different from each other. While not shown, robotic arm assembly (110) may include additional adjustable arm supports (114) coupled with additional robotic arms similar to table-based robotic system (28). First, second, and third robotic arms (112a-c) and adjustable arm support (114) may be positioned and used within a sterile field during medical procedures.

Each robotic arm (112a-c) may move simultaneously and independently relative to the other robotic arms (112a-c). For example, first robotic arm (112a), second robotic arm (112b), and third robotic arm (112c) may each be moved independently relative to each other. First, second, and third robotic arms (112a-c) extend along respective first, second, and third longitudinal axes (LA1, LA2, LA3). First, second, and third robotic arms (112a-c) each include respective proximal and distal ends. As shown, first robotic arm (112a) includes a first connecting portion (116a) defining the proximal end, a first joint (118a), and a first instrument driver (120a) defining the distal end. Similarly, second robotic arm (112b) includes a second connecting portion (116b) defining the proximal end, a second joint (118b), and a second instrument driver (120b) defining the distal end. Third robotic arm (112c) includes a third connecting portion (116c) defining the proximal end, a third joint (118c), and a third instrument driver (120c) defining the distal end. Additional joints are also envisioned.

One or more adjustable arm supports (114) (each supporting one or more robotic arms (112a-c)) may be positioned near a patient platform or table (e.g., tables (16, 34)). In some versions, two adjustable arm supports (114) may be attached to a column supporting table (16, 34), with adjustable arm supports (114) disposed on each side of table (16, 34). Each adjustable arm support (114) may support one or more of robotic arms (112a-c). Adjustable arm support (114) may be adjustable to move relative to table (16, 34) to support and position first, second, and third robotic arms (112a-c). In some examples, adjustable arm support (114) may move in at least one degree of freedom, such as vertically relative to the table (16, 34). In addition to vertical movement, adjustable arm support (114) may also be capable of additional degrees of freedom via tilt or horizontal translation. In some examples, adjustable arm support (114) may move downwardly relative to table (16, 34) to store adjustable arm support (114) and first, second, and third robotic arms (112a-c) positioned thereon underneath table (16, 34) when not in use. In some versions, during use, adjustable arm support (114) may move upwardly relative to table (16, 34) to allow first, second, and third robotic arms (112a-c) to position first, second, and third robotic arms (112a-c) to access to a patient positioned on table (16, 34). In some examples, adjustable arm support (114) is capable of at least three or four degrees of freedom.

As shown in FIG. 7, adjustable arm support (114) includes a bar (122). Bar (122) includes opposing first and second ends (124, 126) and a track (128) (see FIG. 8) operatively connected with first, second, and third connecting portions (116a-c). As shown, bar (122) extends transversely relative to first, second, and third robotic arms (112a-c). First, second, and third connecting portions (116a-c) may allow first, second, and third robotic arms (112a-c) to linearly translate along the length of the adjustable arm support (114), between first end (124) of bar (122) and second end (126) of bar (122) of adjustable arm support (114).

In addition to the foregoing, first, second, and third robotic arms (112a-c), and/or adjustable arm support (114) may be configured and operable in accordance with at least some of the teachings of U.S. Pub. No. 2020/0085516, entitled “Systems and Methods for Concomitant Medical Procedures,” published on Mar. 19, 2020; and/or U.S. Pat. No. 10,500,001, entitled “Surgical Robotics System,” issued on Dec. 10, 2019, the disclosure of each which is incorporated by reference herein, in its entirety.

B. Example of Sterile Drape Assembly

Before, during, and after surgery, first, second, and third robotic arms (112a-c) and bar (122) may be draped in a sterile manner for use in medical procedures. For example, a sterile drape may be used to protect first, second, and third robotic arms (112a-c) and/or bars (122). Use of such a sterile drape may prevent a need for robotic arms (112a-c) and/or bars (122) to be sterilized. The sterile drape may be arranged into a compact arrangement for storage and transport before use; and unfurl in a specific sequence during installation, allowing the user to apply the sterile drape without contaminating the sterile surfaces of the sterile drape before the robotic operation starts.

To maintain a small packaging size, sterile instrument adapters may be tucked into frames of the sterile drape. Before the operation and to prevent contamination, when a user picks up the sterile drape, it may be beneficial for sterile adapters to remain within a frame of the sterile drape and not fall outside the sterile drape from either the top or the bottom. For example, the sterile drape may be disposed around the sterile adapters, and the bottoms of the sterile adapters may be taped in place with breakable tear labels. To release the sterile drape from the bottom, the user severs or otherwise breaches the breakable tear labels on the bottom.

While breakable tear labels may hold the telescoping fold of the drape and/or the sterile adapters in place while the sterile drape is placed onto robotic arm assembly (110), the user may need to manually sever or otherwise breach the breakable tear labels to release the sterile drape onto robotic arm assembly (110). This make may make use of the breakable tear labels cumbersome and/or time consuming. For example, severing breakable tear labels that hold the telescoping fold of the sterile drape in place may constitute an additional task that adds further complexity to the draping process. Moreover, some users may be uncomfortable simply releasing the sterile drape with the severed breakable tear labels. In addition, some breakable tear labels that hold the sterile adapters in place may not be easily accessible (e.g., may be located underneath the sterile adapters) and may be overlooked by the user when releasing the sterile drape, which may confuse or frustrate the user. Reducing the number of breakable tear labels so that the user does not sever or otherwise breach as many breakable tear labels may result in the breakable tear labels not being strong enough to prevent the sterile adapters from falling out the bottom. There is also a possibility that the sterile adapters may fall outside of the sterile drape during the unfolding process with the taping method. For example, the breakable tear labels may allow some movement of the sterile adapters before the movement is restricted by the breakable tear labels.

Separately, sleeves of the sterile drape may be folded with a telescoping pattern (i.e., up and down around the outside of the sleeve in a radial pleat). However, the telescopic pattern may be difficult to retain because other features (e.g., sterile adapters) are attached to the sleeves during the unfolding process. These features may cause the sleeve to unravel too quickly due to the weight of the sterile adapters, which may result in the sterile adapter bouncing off the sterile drape. Secondly, the telescoping pattern may limit the ability of the manufacturer to produce more complex drapes, where sealing operations are difficult to perform while maintaining the telescoping folds. In some versions, telescoping pattern may be taped in place using tear labels after folding to ensure they do not come undone during the rest of the manufacturing operation. Alternatively, or in addition to breakable tear labels, removable tape may hold the telescoping pattern in place which may be subsequently removed during manufacture. However, this increases the number of tear labels to be manually severed or otherwise breached to suitably position the sterile drape on table-based robotic system (28).

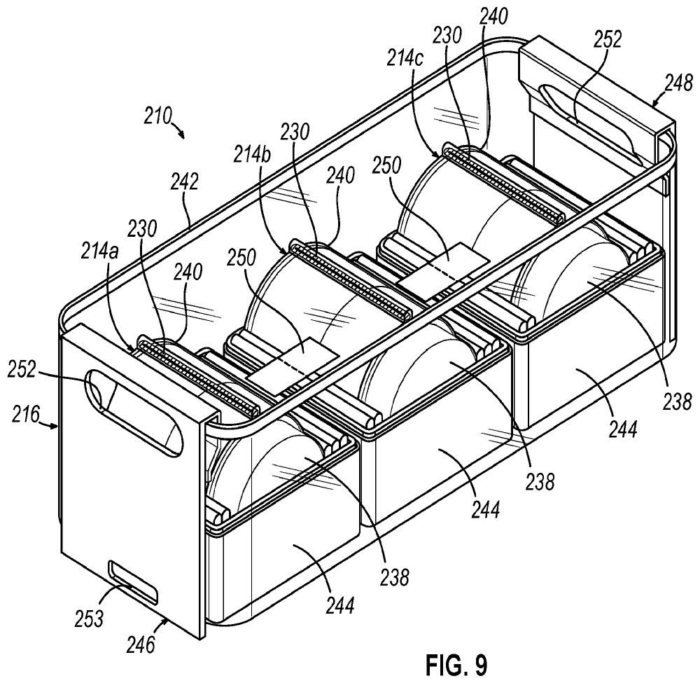

In view of the foregoing, it may be desirable to provide a sterile drape assembly that avoids the shortcomings described above without adversely affecting performance of the sterile drape assembly. FIGS. 8-15 show an example of a sterile drape assembly (210) that may achieve these ends. FIG. 8 shows sterile drape assembly (210) in a deployed configuration covering robotic arm assembly (110) of FIG. 7. FIGS. 9-11 show sterile drape assembly (210) in a packaged configuration prior to deployment. Sterile drape assembly (210) of this example includes a sterile drape (212), sterile adapters (214a-c), and a frame assembly (216). As shown in FIG. 8, sterile drape (212) includes first, second, and third sleeves (218a-c), and an adjustable arm cover (220) that includes a bar cover (222). Sterile drape assembly (210) may include additional sleeves for use with additional robotic arms; and additional adjustable arm covers for use with additional adjustable arms. While not shown, in some versions, a single sleeve of sterile drape (212) may accommodate multiple robotic arm supports. Sterile drape (212) may be disposable, such that sterile drape (212) is only used for a single surgical procedure and then disposed of. In some versions, sterile drape (212) may be formed from thin flexible plastic.

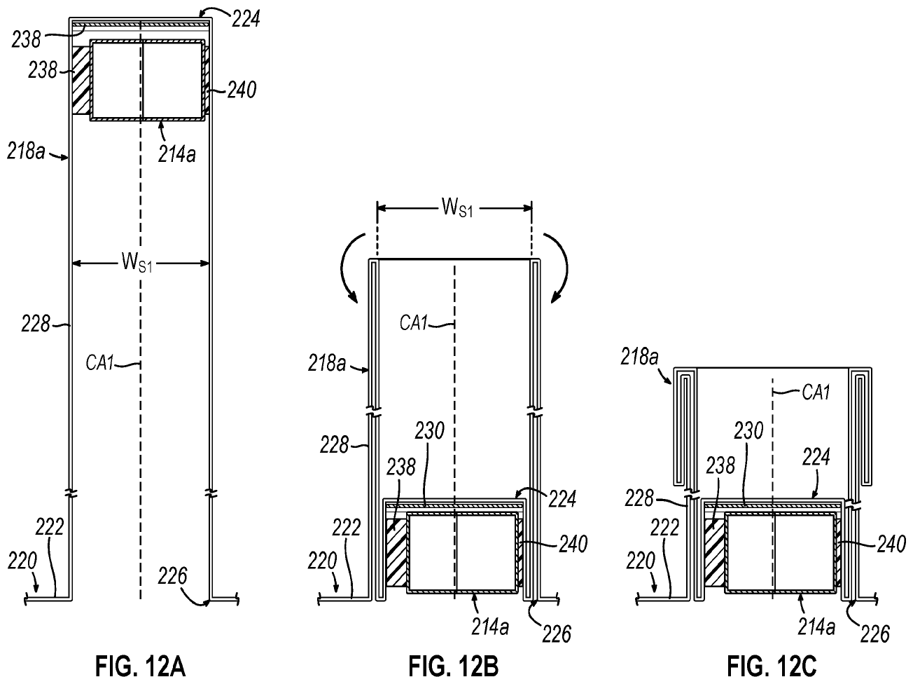

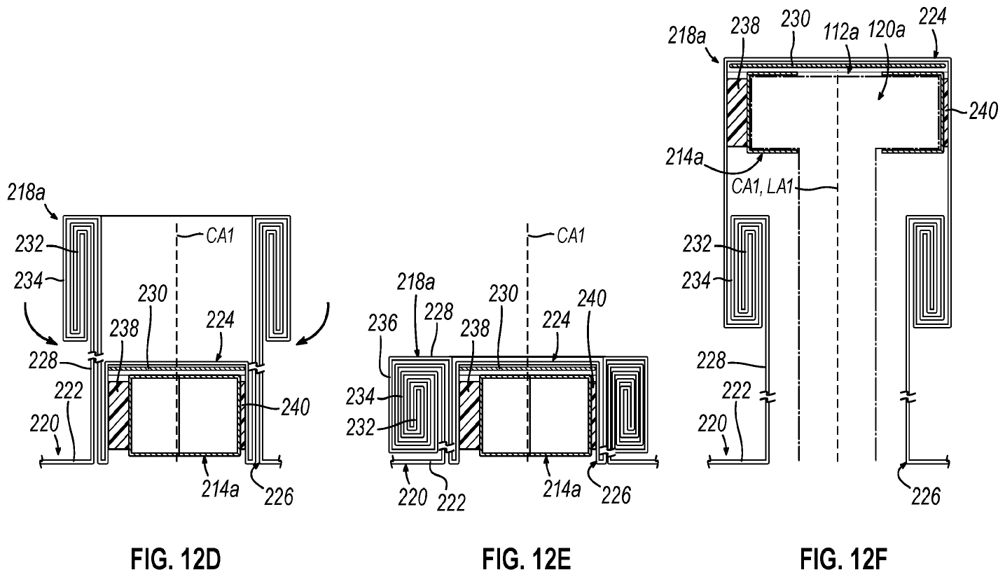

As shown in FIG. 8, first sleeve (218a) is configured to extend over and accommodate first robotic arm (112a). First sleeve (218a) includes a first end (224), a second end (226), and an intermediate portion (228) disposed between first and second ends (224, 226). Second end (226) of first sleeve (218a) is disposed opposite first end (224). In some versions, first end (224) may be a distal end and second end (226) may be a proximal end. First end (224) is coupled with the first sterile adapter (214a). First end (224) is substantially enclosed and may include elastic (230). First sleeve (218a) defines a first central axis (CA1) that extends longitudinally between first and second ends (224, 226) of first sleeve (218a). First sleeve (218a) includes at least one longitudinal seal (227) extending parallel to first central axis (CA1). As will be described in greater detail with reference to FIGS. 12A-12E, intermediate portion (228) includes at least one roll (with first, second, and third rolls (232, 234, 236) being shown).

Similar to first sleeve (218a), second sleeve (218b) is configured to extend over and accommodate second robotic arm (112b); and third sleeve (218c) is configured to extend over and accommodate third robotic arm (112c). Second and third sleeves (218b-c) each include a first end (224), a second end (226), and intermediate portion (228) disposed between first and second ends (224, 226). Second sleeve (218b) defines a second central axis (CA2) that extends longitudinally between first and second ends (224, 226) of second sleeve (218b). Similarly, third sleeve (218c) defines a third central axis (CA3) that extends longitudinally between first and second ends (224, 226) of third sleeve (218c). Similar to first sleeve (218a), intermediate portions (228) of second and third sleeves (218b-c) each include at least one roll (e.g., first, second, and third rolls (232, 234, 236)) in the packaged configuration. As shown in FIG. 8, first central axis (CA1) of first sleeve (218a) is coaxial with longitudinal axis (LA1) of first robotic arm (112a), second central axis (CA2) of second sleeve (218b) is coaxial with longitudinal axis (LA2) of second robotic arm (112b), and third central axis (CA3) of third sleeve (218c) is coaxial with longitudinal axis (LA3) of third robotic arm (112c). First, second, and third central axes (CA1, CA2, CA3) are laterally spaced apart from one another.

First sterile adapter (214a) is configured to be received by first robotic arm (112a). First sterile adapter (214a) is configured to couple with first instrument driver (120a) of first robotic arm (112a). Similarly, second sterile adapter (214b) is configured to be received by second instrument driver (120b) of second robotic arm (112b). Third sterile adapter (214c) is configured to be received by third instrument driver (120c) of third robotic arm (112c). First, second, and third sterile adapters (214a-c) each include first and second adapter portions (238, 240). First adapter portion (238) is configured to couple with a first side (130) of a corresponding one of first, second, and third instrument drivers (120a-c), also referred to as an instrument hub, of robotic arms (112a-c). Second adapter portion (240) is configured to couple with a second side (132) of a corresponding one of first, second, and third instrument drivers (120a-c). Sterile drape assembly (210) may include additional sterile adapters for use with additional robotic arms.

Adjustable arm cover (220) is coupled with first, second, and third sleeves (218a-c) and is configured to extend over adjustable arm support (114). Adjustable arm cover (220) defines an arm support cover width (W_{AC}); and first, second, and third sleeves (218a-c) define respective first, second, and third sleeve width (W_{S1}, W_{S2}, W_{S3}). Adjustable arm cover width (W_{AC}) is greater than individual first, second, and third sleeve widths (W_{S1}, W_{S2}, W_{S3}). As shown, bar cover (222) of adjustable arm cover (220) is coupled with first, second, and third sleeves (218a-c); and is configured to extend over bar (122). Particularly, bar cover (222) is coupled with respective second ends (226) of first, second, and third sleeves (218a-c). Bar cover (222) includes an elastic band (242).

FIGS. 9-11 show sterile drape assembly (210) with sterile drape (212). As shown, sterile drape assembly (210) includes first, second, and third sterile adaptors (214a-c). Sterile drape assembly (210) further includes individual sleeve frames (244) opposing first and second side members (246, 248), with breakable tear labels (250) connecting adjacent first, second, and third sterile adaptors (214a-c). Breakable tear labels (250) may couple together at least one of first sleeve (218a) or first sterile adapter (214a) with at least one of second sleeve (218b) or second sterile adapter (214b). First and second side members (246, 248) include respective hand apertures (252) configured to be gripped by the user; and rotation apertures (253) configured to couple with first and second ends (124, 126) of bar (122).

1. Example of Method of Manufacturing

FIGS. 12A-12E show different stages in an example of a method of manufacturing sterile drape assembly (210). While FIGS. 12A-12E are shown and described with reference to first sleeve (218a), the stages shown in FIGS. 12A-12E may also be used with respect to other sleeves (e.g., second sleeve (218b), third sleeve (218c), etc.) of sterile drape (212).

FIG. 12A shows a schematic cross-sectional side elevational view of first sleeve (218a) of sterile drape (212) after a first manufacturing stage. As shown, first sterile adapter (214a) is already coupled with first end (224) of first sleeve (218a). However, if desired, first sterile adapter (214a) may be coupled with first end 224) of first sleeve (218a) after inverting of intermediate portion and/or formation of rolls (232, 234, 236) as described below. First end (224) is spaced away from second end (226), though ends (224, 226) are coaxially aligned along first central axis (CA1).

As shown in FIG. 12A, arm support cover (220) is coupled with second end (226) of first sleeve (218). At least one sealing process may be performed to couple first, second, and third sleeves (218a-c) with adjustable arm cover (220). In some versions, the sealing process(es) may be performed prior to inverting first end (224) towards second end (226) and rolling intermediate portion (228) as described below. Coupling of bar cover (222) with second ends (226) of first, second, and third sleeves (218a-c) may include heat sealing bar cover (222) with second end (226) of first, second, and third sleeves (218a-c). However, other coupling processes are also envisioned.

FIG. 12B schematically shows first sleeve (218a) of sterile drape (212) of FIG. 12A, but after a second manufacturing stage. As shown, first end (224) is inverted toward second end (226) so that first end (224) is disposed generally adjacent to second end (226), with first and second ends (224, 226) being separated by a distance that is about equal to the diameter of first sterile adapter (214a). As shown when comparing FIG. 12A with FIG. 12B, first end (224) of first sleeve (218a) is inverted into second end (226). As shown in FIG. 12B, about half of first sleeve (218a) is inverted, such that first end (224) is disposed adjacent the connection between first sleeve (218a) and bar cover (222). First sleeve (218a) includes distal and proximal portions. The distal portion terminates at first end (224) connected to first sterile adapter (214a). The proximal portion terminates at second end (226) opposite first end (224). In the packaged configuration, the distal portion of first sleeve (218a) is inverted toward the proximal portion. The distal portion is positioned radially inwardly, toward first central axis (CA1), relative to the proximal portion.

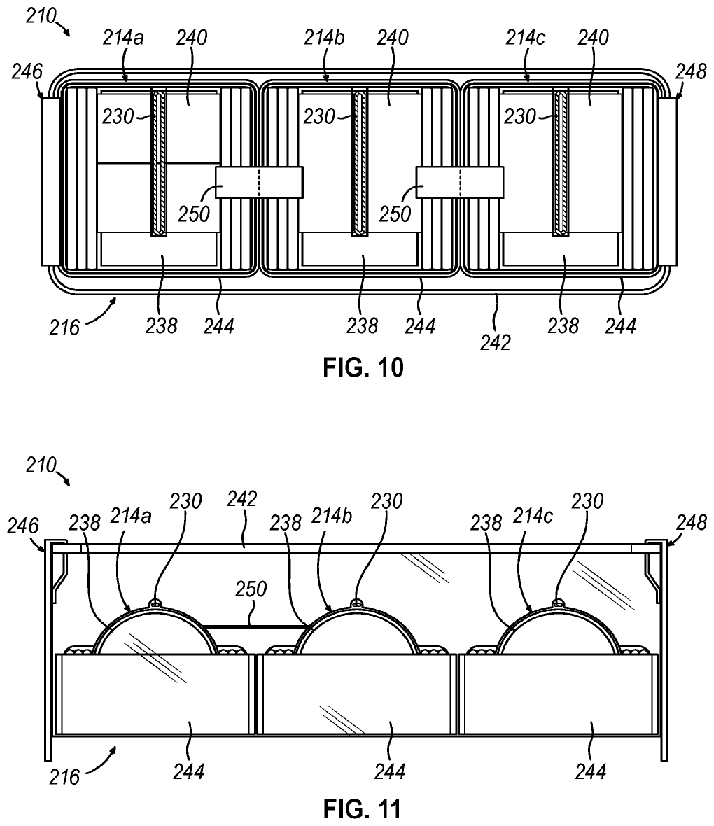

FIG. 12C schematically shows first sleeve (218a) of sterile drape (212) of FIG. 10B, but after a third manufacturing stage. Intermediate portion (228) may be rolled to form at least one roll. First roll (232) is configured to resist unrolling. Intermediate portion (228) may be rolled outwardly away from first central axis (CA1) of first sleeve (218a) towards first and second ends (224, 226), so that intermediate portion (228) approaches first and second ends (224, 226). First roll (232) of first sleeve (218a) is positioned away from first central axis (CA1). FIG. 12D schematically shows first sleeve (218a) of sterile drape (212) of FIG. 12C, but after a fourth manufacturing stage. Intermediate portion (228) includes a second roll (234). First and second rolls (232, 234) are in contact with each other. Like first roll (232), second roll (234) is positioned radially outwardly relative to first central axis (CA1). In addition, second roll (234) is positioned radially outwardly from first roll (232).

FIG. 12E schematically shows first sleeve (218a) of sterile drape (212) of FIG. 12D, but after a fifth manufacturing stage. Intermediate portion (228) includes a third roll (236). Second and third rolls (234, 236) are in contact with each other. Like first and second rolls (232, 234), third roll (236) is positioned radially outwardly relative to first central axis (CA1). In addition, third roll (236) is positioned radially outwardly from second roll (234). While first, second, and third rolls (232, 234, 236) are shown, more or fewer rolls are also envisioned.

In the present example, through the process described above with reference to FIGS. 12A-12E, a uniform roll fold is made around the outside of folded first sleeve (218a). First, second, and third rolls (232, 234, 236) may be refolded if any one of first, second, and third rolls (232, 234, 236) come undone; or as otherwise desired. Rolls (232, 234, 236) are different than the telescoping pattern described above and provide additional benefits. For example, using a roll fold, an entirety of sterile drape (212) may be manufactured prior to folding. As a result, intermediate sleeve material (e.g., intermediate portion (228)) is managed, and folding of rolls (232, 234, 236) may be performed after sealing the open end (e.g., first end (224)) of sterile drape (212). Rolls (232, 234, 236) allow a user to rework sterile drape (212) after sterile drape (212) has become unfolded, lowering the risk of scrap if sterile drape (212) becomes unfolded prematurely. Rolls (232, 234, 236) may enable faster or more complicated operations at first and second ends (224, 226) of sterile drape (212) since the user does not need maintain the previously obtained folds together while performing other manufacturing operations.

While FIGS. 12A-12E are provided in the context of first sleeves (218a), other sleeves (218b-c) may be prepared in a similar fashion. For instance, in the present example, rolls (232, 234, 236) of second sleeve (218b) are rolled away from second central axis (CA2) and toward first and second ends (224, 226) of second sleeve (218b) using the same process described above with reference to FIGS. 12A-12E. Similarly, rolls (232, 234, 236) of third sleeve (218c) are rolled away from third central axis (CA3) and toward first and second ends (224, 226) of second sleeve (218b) using the same process described above with reference to FIGS. 12A-12E. After each sleeve (218a-218c) achieves the state shown in FIG. 12E, sterile drape assembly (210) may be placed in a packaged configuration as described in greater detail below with reference to FIG. 13.

FIG. 12F shows first sleeve (218a) of sterile drape (212) of FIG. 12E after first robotic arm (112a) partially unrolls sterile drape (212) during application of sterile drape (212). As shown, first sleeve (218a) includes a generally tubular cover with a wider first end (224) to accommodate instrument driver (120a). Further details of how sterile drape assembly (210) may be coupled with robotic arm assembly (110) will be described in greater detail below.

2. Example of Locking Assembly

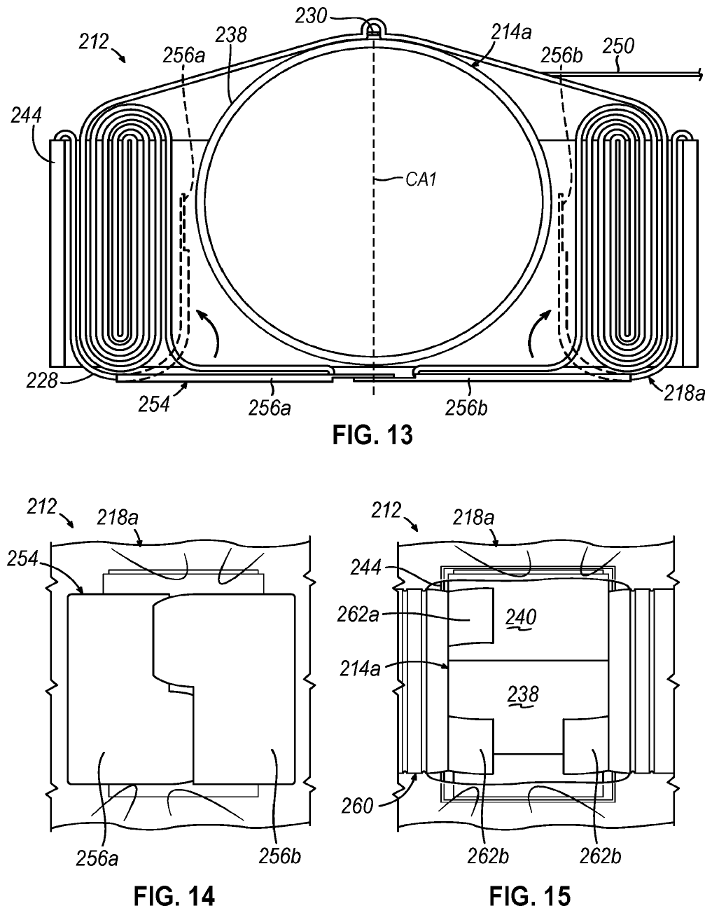

FIGS. 13-14 show a portion of sterile drape assembly (210) that includes an example of a locking assembly (254) in a closed configuration, with an open configuration being shown in phantom. While FIGS. 13-14 show a single locking assembly (254), it is envisioned that sterile drape assembly (210) may include multiple locking assemblies (254). For example, separate locking assemblies (254) may be used for each of first, second, and third sterile adapters (214a-c). Locking assembly (254), also referred to as a trapdoor assembly, is oriented such that the trapdoor does not open downwardly; but instead only opens upwardly.

Locking assembly (254) includes first and second flaps (256a-b) that are configured to couple together to resist separation in a first direction; but allow separation in a second direction that is opposite to the first direction. FIG. 14 shows a bottom view of locking assembly (254) where first and second flaps (256a-b) are interlocked together in the closed configuration to support weight of respective sterile adapters (214a-c). Each flap (256a-b) may be supported from below by another feature. This may prevent locking assembly (254) from opening downwardly, even with the weight of one of first, second, and third sterile adapters (214a-c) sitting on top. First and second flaps (256a-b) remain locked in place when sterile drape (212) is unpackaged and transported to robotic arm assembly (110). First and second flaps (256a-b) are positioned within frame assembly (216) adjacent to first sterile adapter (214a) and first sleeve (218a).

When sterile drape assembly (210) is being installed on robotic arm assembly (110), robotic arms (112a-c) force first and second flaps (256a-b) to the open position by pushing flaps (256a-b) upwardly toward respective sterile adapters (214a-c). First and second flaps (256a-b) fold upwardly to allow a respective robotic arm (112a-c) to pass through locking assembly (254). In other words, when the frame assembly (216) travels over robotic arms (112a-c), respective robotic arms (112a-c) push on each locking assembly (254) from below and open each locking assembly (254) in an upward direction. In the open configuration, instrument drivers (120a-c) of robotic arm (112a-c) may fully enter into first, second, and third sleeves (218a-c), without requiring an additional user step to release sterile adapter (214a-c). In other words, first and second flaps (256a-b) automatically release sterile adapters (214a-c), without requiring the user to perform an additional step (e.g., releasing one or more breakable tear labels on the bottom). Each locking assembly (254) thus eliminates a workflow step, is able to support the weight of a corresponding sterile adapter (214a-c), and does not allow sterile adapters (214a-c) to sag beneath sterile drape (212) when sterile drape assembly (210) is picked up by the user. While first and second flaps (256a-b) are shown, more or fewer flaps are also envisioned.

3. Another Example of Locking Assembly

FIG. 15 shows another example of a locking assembly (260) for use with sterile drape assembly (210). Locking assembly (260) of this example may be used instead of locking assembly (254) or in addition to locking assembly (254). FIG. 15 shows first and second locking features (262a-b) of locking assembly (260). Locking features (262a-b) are configured to tuck around sterile adapter (214a-c) and lock sterile adapter (214a-c) in place; but allow robotic arm (214a-c) to penetrate through locking assembly (260). Each set of first and second locking features (262a-b) locks against and supports a corresponding one of first, second, and third sterile adapters (214a-c). While FIG. 15 shows a single locking assembly (260), it is envisioned that sterile drape assembly (210) may include multiple locking assemblies (260). For example, separate locking assemblies (260) may be used for each of first, second, and third sterile adapters (214a-c).

In the present example, first and second locking features (262a-b) are unable to bend around at the hinge and open downwardly because the volume of sterile adapter (214a-c) prevents first and second locking features (262a-b) from doing so. Similarly, sterile adapter (214a-c) is supported by first and second locking features (262a-b) and thereby cannot fall through sleeve frame (244). When robotic arms (112a-c) enter from below, robotic arms (112a-c) push corresponding sterile adapters (214a-c) off first and second locking features (262a-b) by pushing first and second locking features (262a-b) outwardly to allow robotic arms (112a-c) into sterile drape (212). Similar to locking assembly (254), sterile adapter (214a-c) is released automatically during draping.

C. Example of Method of Use

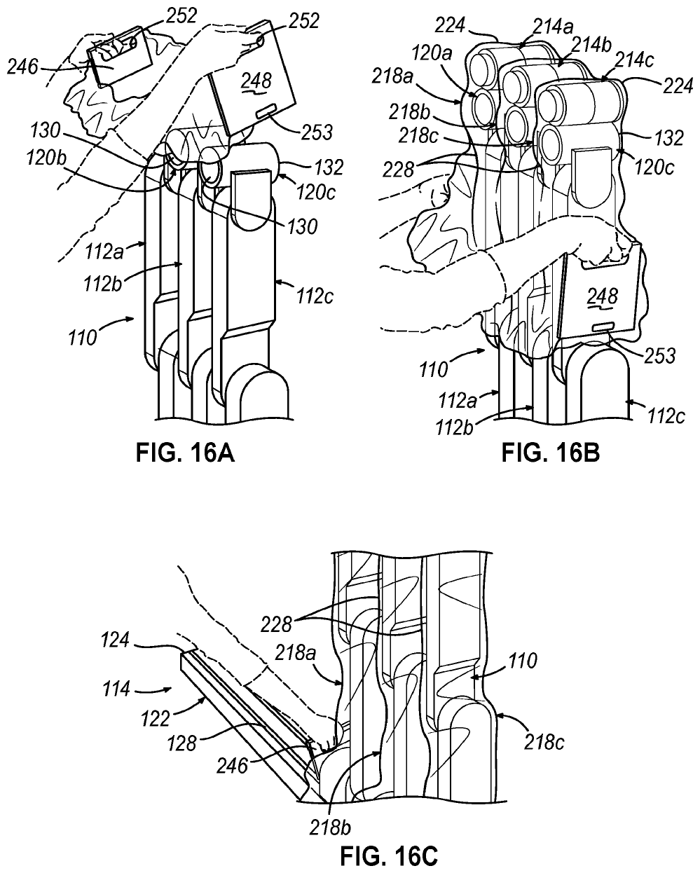

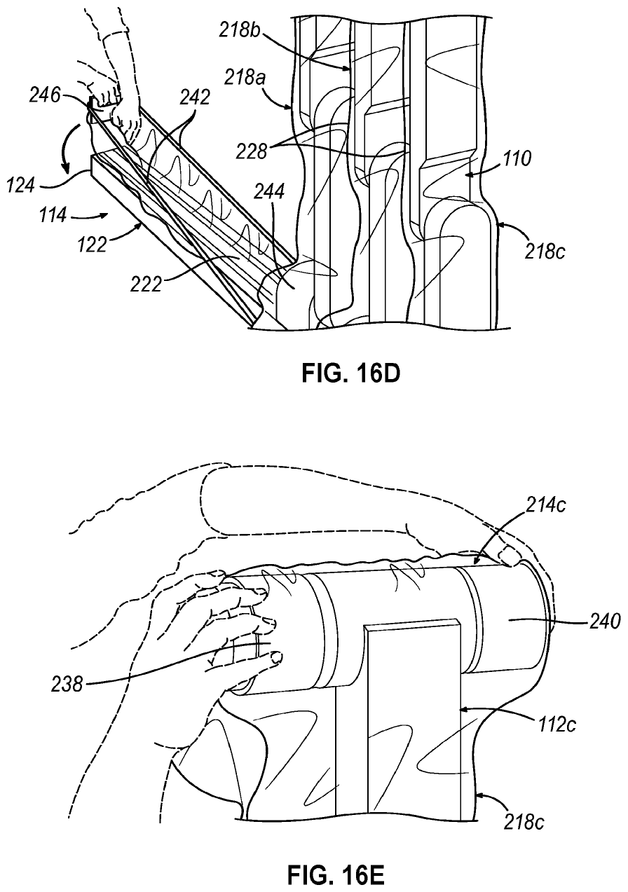

FIGS. 16A-16E show an example of using sterile drape assembly (210) with robotic arm assembly (110). FIG. 16A shows sterile drape assembly (210) being placed over first, second, and third robotic arms (112a-c) of FIGS. 7-8, with a user grasping sterile drape assembly (210) via hand apertures (252). Locking assemblies (254, 260) of sterile drape assembly (210) supports the weight of corresponding sterile adapters (214a-c) during the stage shown in FIG. 16A, such that locking assemblies (254, 260) prevent sterile adapters (214a-c) from falling downwardly during the stage shown in FIG. 16A.

FIG. 16B shows sterile drape assembly (210) being simultaneously moved over first, second, and third robotic arms (112a-c), toward the adjustable arm support of FIG. 7. First, second, and third instrument drivers (120a-c) of first, second, and third robotic arms (112a-c) fully enter into first, second, and third sleeves (218a-c) without a user step to release sterile adapter (214a-c) from below using breakable tear labels using locking assembly (254) and/or locking assembly (260). In other words, instrument drivers (120a, 120b, 120c) bear upwardly against corresponding locking assemblies (254, 260) during the stage shown in FIG. 16B, such that instrument drivers (120a, 120b, 120c) effectively open locking assemblies (254, 260) during the stage shown in FIG. 16B. While still grasping sterile drape assembly (210) via hand apertures (252), the user may continue to urge bar cover (222) downwardly toward first end (124) of bar (122) of adjustable arm support (114). Sterile adapters (214a-c) may remain positioned adjacent to corresponding instrument drivers (120a, 120b, 120c) as the user continues urging bar cover (222) downwardly toward bar (122).

FIG. 16C shows robotic arms (112a-c), sterile drape assembly (210), and adjustable arm support (114) of FIG. 16B, with bar cover (222) of sterile drape assembly (210) being moved toward first end (124) of bar (122) of adjustable arm support (114). At this stage, each robotic arm (112a-c) is completely covered or substantially covered by a corresponding sleeve (218a-c) of sterile drape assembly (210). FIG. 16D shows a perspective view of sterile drape assembly (210) and adjustable arm support (114) of FIG. 16C, with bar cover (222) of sterile drape assembly (210) being rotated over first end (124) of bar (122) of adjustable arm support (114). This may be achieved by the user grasping sterile drape assembly (210) via a hand aperture (252) and thereby pulling bar cover (222) to encompass first end (124) of bar (122) of adjustable arm support (114). Elastic band (242) in bar cover (222) may stretch during this stage. While FIG. 16D only depicts the installation process with respect to first end (124) of bar (122), the same procedure may be carried out in a similar fashion with respect to second end (126) of bar (122). At that point in the process, sterile drape assembly (210) may be covering all robotic arms (112a-c) and the full length of bar (122).

With all robotic arms (112a-c) and the full length of bar (122) being covered by sterile drape assembly (210), a next stage may include coupling of sterile adapters (214a-c) with corresponding instrument drivers (120a-c). To that end, FIG. 16E shows third sterile adapter (214c) of sterile drape assembly (210) being coupled with instrument driver (120c) of robotic arm (112c). The user severs breakable tear labels (250) connecting adjacent first, second, and third sterile adaptors (214a-c) prior to coupling sterile adapters (214a-c) with instrument drivers (120a-c). As noted above, each sterile adapter (214a-c) includes first and second adapter portions (238, 240). First adapter portion (238) couples with first side (130) of instrument driver (120a-c). Second adapter portion (240) couples with second side (132) of instrument driver (120a-c). Adapter portions (238, 240) provide a sterile mechanical coupling between each instrument driver (120a-c) and a corresponding surgical instrument (14), such that instrument drivers (120a-c) may effectively drive corresponding surgical instruments (14) via sterile adapters (214a-c).

In addition to the foregoing, sterile drape assembly (210) may be installed on robotic arm assembly (110) in accordance with at least some of the teachings of U.S. Pub. No. 2021/0153965, entitled “Systems and Methods for Draping a Surgical System,” published on May 27, 2021; U.S. Pub. No. 2021/0153966, entitled “Drape for Arms of a Robotic Surgical System,” published on May 27, 2021.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An assembly comprising: (a) a first sterile adapter configured to be received by a first movable robotic arm of a robotic surgical system; and (b) a sterile drape that includes a first sleeve configured to extend over the first movable robotic arm, the first sleeve comprising: (i) a first end coupled with the first sterile adapter, (ii) a second end disposed opposite the first end, wherein the first end is inverted toward the second end, and (iii) an intermediate portion disposed between the first and second ends, wherein the intermediate portion includes a first roll.

### Example 2

The assembly of Example 1, wherein the first roll is configured to resist unrolling.

### Example 3

The assembly of any of Examples 1 through 2, wherein the first roll is oriented toward the first and second ends.

### Example 4

The assembly of any of Examples 1 through 3, wherein the intermediate portion further comprises a second roll, wherein the first and second rolls are in contact with each other.

### Example 5

The assembly of any of Examples 1 through 4, wherein the first sleeve includes a tubular cover.

### Example 6

The assembly of any of Examples 1 through 6, wherein the first sterile adapter includes a first adapter portion configured to couple with a first side of an instrument hub of the first movable robotic arm.

### Example 7

The assembly of Example 6, wherein the first sterile adapter includes a second adapter portion configured to couple with a second side of the instrument hub of the first movable robotic arm.

### Example 8

The assembly of Example 7, wherein the first and second adapter portions are configured to couple together.

### Example 9

The assembly of any of Examples 1 through 7, wherein the first end is substantially enclosed.

### Example 10

The assembly of any of Examples 1 through 9, wherein the first end includes elastic.

### Example 11

The assembly of any of Examples 1 through 10, further comprising first and second flaps configured to couple together to allow separation in a first direction and resist separation in a second direction that is opposite to the first direction.

### Example 12

The assembly of Example 11, wherein the first sterile adapter is configured to be removably housed adjacent to the first and second flaps.

### Example 13

The assembly of any of Examples 1 through 12, further comprising: (a) a second sterile adapter configured to be received by a second movable robotic arm of the robotic surgical system; and (b) a second sleeve configured to extend over the second movable robotic arm, the second sleeve comprising: (i) a first end coupled with the second sterile adapter, (ii) a second end disposed opposite the first end of the second sleeve, wherein the first end of the second sleeve is inverted toward the second end of the second sleeve, and (iii) an intermediate portion disposed between the first and second ends of the second sleeve, wherein the intermediate portion of the second sleeve includes a first roll.

### Example 14

The assembly of Example 13, the second sleeve being coupled with the first sleeve.

### Example 15

The assembly of any of Examples 13 through 14, the second sleeve being oriented parallel with the first sleeve, the second sleeve being laterally offset from the first sleeve.

### Example 16

The assembly of any of Examples 13 through 15, further comprising a cover extending from the first sleeve to the second sleeve.

### Example 17

The assembly of Example 16, the cover being oriented transversely relative to the first and second sleeves.

### Example 18

The assembly of any of Examples 16 through 17, the cover being configured to cover a robotic arm support of the robotic surgical system, the first and second movable robotic arms being supported by the robotic arm support.

### Example 19

The assembly of Example 18, the robotic arm support comprising an elongate member.

### Example 20

The assembly of Example 19, the elongate member comprising a bar.

### Example 21

The assembly of any of Examples 13 through 20, further comprising a tear label configured to couple together at least one of the first sleeve or the first sterile adapter with at least one of the second sleeve or the second sterile adapter.

### Example 22

The assembly of any of Examples 1 through 21, wherein the first sleeve defines a first central axis that extends longitudinally between the first and second ends of the first sleeve, wherein the first roll of the first sleeve is oriented away from the first central axis and toward the first and second ends of the first sleeve.

### Example 23

The assembly of any of Example 22, wherein the first sleeve includes at least one longitudinal seal extending parallel to the first central axis.

### Example 24

The assembly of any of Examples 1 through 23, further comprising the first movable robotic arm, wherein the first sterile adapter is coupled with an end of the first movable robotic arm, wherein the first sleeve extends along a length of the first movable robotic arm.

### Example 25

The assembly of Example 24, wherein a central axis of the first sleeve is configured to extend parallel to a longitudinal axis of the first movable robotic arm.

### Example 26

The assembly of any of Examples 1 through 25, further comprising: (a) the first movable robotic arm; (b) a second movable robotic arm; and (c) an adjustable arm support, wherein the first and second movable robotic arms are coupled with the adjustable arm support.

### Example 27

The assembly of Example 26, the sterile drape further including an arm support cover coupled with the first sleeve and configured to extend over the adjustable arm support.

### Example 28

The assembly of Example 27, wherein the arm support cover defines an arm support cover width, wherein the first sleeve defines a first sleeve width, wherein the arm support cover width is greater than the first sleeve width.

### Example 29

The assembly of any of Examples 27 through 28, wherein the adjustable arm support includes a bar, wherein the bar extends transversely relative to the first movable robotic arm, wherein the arm support cover is coupled with the first sleeve and configured to extend over the bar.

### Example 30

A sterile drape assembly comprising: (a) a sterile adapter configured to couple to an instrument interface of a movable robotic arm of a robotic surgical system; and (b) a sterile drape comprising a sleeve configured to extend over the movable robotic arm, the sleeve comprising: (i) a first portion terminating at a first end connected to the sterile adapter, and (ii) a second portion terminating at a second end opposite the first end, wherein, in a packaged configuration, the first portion of the sleeve is inverted toward the second portion of the sleeve, and an intermediate portion of the sleeve between the first and second ends is rolled.

### Example 31

The sterile drape assembly of Example 30, further comprising an arm support cover coupled with the sleeve and configured to extend over an adjustable arm support that is coupled with the movable robotic arm.

### Example 32

An assembly comprising: (a) a first sterile adapter configured to be received by a first movable robotic arm of a robotic surgical system; and (b) a first sleeve coupled with the first sterile adapter; and (c) first and second flaps that collectively form a trapdoor, wherein the first and second flaps are configured to couple together to resist separation in a first direction and support the first sterile adapter, wherein the first and second flaps are further configured to allow separation in a second direction that is opposite to the first direction, the first and second flaps being positioned adjacent to the first sterile adapter and the first sleeve.

### Example 33

The assembly of Example 32, wherein the first and second flaps are configured to fold in the first direction as the assembly is manually moved in the second direction.

### Example 34

The assembly of any of Examples 32 through 33, further comprising a frame assembly configured to provide rigidity to the assembly.

### Example 35

A method of manufacturing an assembly, the assembly comprising a first sterile adapter and a sterile drape configured to be coupled with the sterile drape, wherein the sterile drape includes a first sleeve configured to extend over a first movable robotic arm of a robotic surgical system, wherein the first sleeve includes a first end and a second end disposed opposite the first end, the method comprising: (a) inverting the first end toward the second end to form an intermediate portion disposed between the first and second ends of the first sleeve; and (b) rolling the intermediate portion to form a first roll.

### Example 36

The method of Example 35, further comprising coupling the first sterile adapter with the first sleeve.

### Example 37

The method of any of Examples 35 through 36, further comprising performing at least one sealing process prior to the act of rolling the intermediate portion.

### Example 38

The method of any of Examples 35 through 37, wherein the act of rolling the first intermediate portion further comprises rolling the first intermediate portion outwardly away from a first central axis of the first sleeve toward the first and second ends so that the first intermediate portion approaches the first and second ends.

### Example 39

The method of any of Examples 35 through 38, further comprising coupling an arm support cover with a second end of the first sleeve, wherein an arm support cover width of the arm support cover is greater than a first sleeve width of the first sleeve.

### Example 40

The method of Example 38, wherein the arm support cover includes a bar cover, the method further comprising coupling the bar cover with a second end of the first sleeve, wherein a bar cover width of the bar cover is greater than a first sleeve width of the first sleeve.

### Example 41

The method of Example 34, wherein the act of coupling the bar cover with the second end of the first sleeve comprises heat sealing the bar cover with the second end of the first sleeve.

### Example 42

The method of any of Examples 35 through 41, the assembly comprising a second sterile adapter, wherein the sterile drape includes a second sleeve configured to extend over a second movable robotic arm of the robotic surgical system, wherein the second sleeve includes a first end and a second end disposed opposite the first end of the second sleeve, the method further comprising: (a) coupling the second sterile adapter with the second sleeve; (b) coupling the second sleeve with the sterile drape; (c) inverting the first end of the second sleeve toward the second end of the second sleeve to form an intermediate portion disposed between the first and second ends of the second sleeve; and (d) rolling the intermediate portion of the second sleeve to form a first roll in the second sleeve.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An assembly comprising:
(a) a first sterile adapter configured to be received by a first movable robotic arm of a robotic surgical system; and
(b) a sterile drape that includes a first sleeve configured to extend over the first movable robotic arm, the first sleeve comprising:
(i) a first end coupled with the first sterile adapter,
(ii) a second end disposed opposite the first end, wherein the first end is inverted toward the second end, and
(iii) an intermediate portion disposed between the first and second ends, wherein the intermediate portion includes a first roll.

2. The assembly of claim 1, wherein the first roll is configured to resist unrolling.

3. The assembly of claim 1, wherein the first roll is oriented toward the first and second ends.

4. The assembly of claim 1, wherein the intermediate portion further comprises a second roll, wherein the first and second rolls are in contact with each other.

5. The assembly of claim 1, wherein the first sleeve includes a tubular cover.

6. The assembly of claim 1, wherein the first sterile adapter includes a first adapter portion configured to couple with a first side of an instrument hub of the first movable robotic arm.

7. The assembly of claim 6, wherein the first sterile adapter includes a second adapter portion configured to couple with a second side of the instrument hub of the first movable robotic arm.

8. The assembly of claim 7, wherein the first and second adapter portions are configured to couple together.

9. The assembly of claim 1, wherein the first end is substantially enclosed.

10. The assembly of claim 1, wherein the first end includes elastic.

11. The assembly of claim 1, further comprising first and second flaps configured to couple together to allow separation in a first direction and resist separation in a second direction that is opposite to the first direction.

12. The assembly of claim 11, wherein the first sterile adapter is configured to be removably housed adjacent to the first and second flaps.

13. The assembly of claim 1, further comprising:
(a) a second sterile adapter configured to be received by a second movable robotic arm of the robotic surgical system; and
(b) a second sleeve configured to extend over the second movable robotic arm, the second sleeve comprising:
(i) a first end coupled with the second sterile adapter,
(ii) a second end disposed opposite the first end of the second sleeve, wherein the first end of the second sleeve is inverted toward the second end of the second sleeve, and
(iii) an intermediate portion disposed between the first and second ends of the second sleeve, wherein the intermediate portion of the second sleeve includes a first roll.

14. The assembly of claim 13, further comprising a tear label configured to couple together at least one of the first sleeve or the first sterile adapter with at least one of the second sleeve or the second sterile adapter.

15. The assembly of claim 1, wherein the first sleeve defines a first central axis that extends longitudinally between the first and second ends of the first sleeve, wherein the first roll of the first sleeve is oriented away from the first central axis and toward the first and second ends of the first sleeve.

16. The assembly of claim 1, further comprising the first movable robotic arm, wherein the first sterile adapter is coupled with an end of the first movable robotic arm, wherein the first sleeve extends along a length of the first movable robotic arm.

17. The assembly of claim 1, further comprising:
(a) the first movable robotic arm;
(b) a second movable robotic arm; and
(c) an adjustable arm support, wherein the first and second movable robotic arms are coupled with the adjustable arm support.

18. The assembly of claim 17, the sterile drape further including an arm support cover coupled with the first sleeve and configured to extend over the adjustable arm support.

19. A sterile drape assembly comprising:
(a) a sterile adapter configured to couple to an instrument interface of a movable robotic arm of a robotic surgical system; and
(b) a sterile drape comprising a sleeve configured to extend over the movable robotic arm, the sleeve comprising:
(i) a first portion terminating at a first end connected to the sterile adapter, and
(ii) a second portion terminating at a second end opposite the first end, wherein, in a packaged configuration, the first portion of the sleeve is inverted toward the second portion of the sleeve, and an intermediate portion of the sleeve between the first and second ends is rolled.

20. An assembly comprising:
(a) a first sterile adapter configured to be received by a first movable robotic arm of a robotic surgical system; and
(b) a first sleeve coupled with the first sterile adapter; and
(c) first and second flaps that collectively form a trapdoor, wherein the first and second flaps are configured to couple together to resist separation in a first direction and support the first sterile adapter, wherein the first and second flaps are further configured to allow separation in a second direction that is opposite to the first direction, the first and second flaps being positioned adjacent to the first sterile adapter and the first sleeve.
